(19) United States
(12) Patent Application Publication
(10) Pub. No.: US 2018/0230081 A1
(43) Pub. Date: Aug. 16, 2018

(54) PROCESS FOR PREPARING ISOPHORONE AMINO ALCOHOL (IPAA)

(71) Applicant: Evonik Degussa GmbH, Essen (DE)

(72) Inventors: Alexander Martin Rüfer, Recklinghausen (DE); Anne Rittsteiger, Meltingen (CH); René Poss, Hanau (DE); Meike Roos, Büdingen (DE); Stefan Röder, Sinntal (DE); Monika Berweiler, Maintal (DE)

(21) Appl. No.: 15/894,187

(22) Filed: Feb. 12, 2018

(30) Foreign Application Priority Data
Feb. 15, 2017 (DE) 102017202404.6

(51) Int. Cl. C07C 213/00; B01J 23/75; B01J 21/02; B01J 35/04; B01J 35/10

(52) U.S. Cl. CPC C07C 213/00; B01J 23/75; B01J 21/02; C07C 2601/14; B01J 35/1076; B01J 35/1014; B01J 35/1019; B01J 35/04

(57) ABSTRACT

The present invention relates to an improved process for preparing 3-(aminomethyl)-3,5,5-trimethylcyclohexanol, called isophorone amino alcohol (IPAA) hereinafter. In particular, the present invention is directed to a one-stage process for preparing 3-aminomethyl-3,5,5-trimethylcyclohexanol by hydrogenation of isophoronenitrile (IPN) with hydrogen over a catalyst, in the presence or absence of solvents, wherein the catalyst has certain properties.

The present invention relates to an improved process for preparing 3-aminomethyl-3,5,5-trimethylcyclohexanol from isophoronenitrile, called isophorone amino alcohol (IPAA) hereinafter.

IPAA is an important intermediate in various fields of use. For example, it serves as a precursor of pharmacological products, especially in the field of influenza prophylaxis (WO2011/095576). Further applications include use in polymers, anticorrosives and stabilizers (DE1229078).

The existing processes feature comparatively complicated reaction conduct and low yields.

Pandey et al. (Indian Journal of Chemistry, vol. 43B, 2004, 2705-2707) describes a synthesis route to IPAA proceeding from isophoronenitrile (IPN). This proceeds over several stages: first of all, the corresponding amide is formed via hydrolysis under basic conditions. Then the keto function is reduced by means of cyanoborohydride and the amide is reduced by means of lithium aluminium hydride.

A similar procedure is described in applications WO2011/094953 and WO2011/095576, but the keto and nitrile groups of IPN are reduced here exclusively with lithium aluminium hydride in THF under reflux. A disadvantage is considered to be that the abovementioned processes envisage the stoichiometric use of reducing agents that are difficult to prepare and hence costly. Moreover, the synthesis is effected over several stages and is thus laborious.

Application DE1229078 describes the preparation of IPAA from IPN with the aid of ammonia and hydrogen over a Fischer-Tropsch catalyst under very high partial hydrogen pressure. However, the relatively high reaction temperatures required here in the case of gamma-keto nitriles (such as IPN) promote the elimination of hydrogen cyanide (HCN). This circumstance leads to a reduction in yield and is therefore disadvantageous. A further disadvantage is considered to be that HCN has a highly toxic effect on organisms (risk on release) and typically contributes to catalyst deactivation. Moreover, there is substantial formation of dimers because of the reaction of the amine group formed with as yet incompletely reduced keto group. This likewise constitutes a loss of yield based on the desired IPAA product.

It is also known from EP 42 119, EP 659 734, EP 503 246, WO 2012/076315 that IPAA forms only undesirably as a by-product in the conventional hydrogenation of IPN, which is also referred to as a disadvantage. Here too, reference is made to the elimination of hydrogen cyanide (HCN). These disadvantages can be avoided through the use of pelletized Raney-type catalysts from EP 3050870.

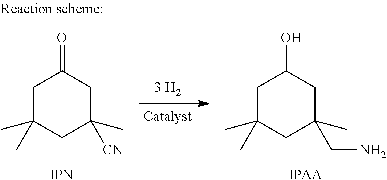

The underlying object was to find a process for preparing 3-aminomethyl-3,5,5-trimethylcyclohexanol (IPAA) by hydrogenation of isophoronenitrile (IPN) with the aid of hydrogen on a catalyst, wherein the catalyst should firstly have as low a bulk density as possible, and secondly have a high activity for the specified hydrogenation reaction.

If Raney cobalt catalysts of the pellet type according to EP 3050870 are used for the preparation of IPAA by hydrogenation of IPN, they typically have a bulk density of more than 1.5 kg/l. In the case of customary reactor sizes of 5 to 50 m³, this corresponds to the required amount of cobalt per catalyst charge of 8 to 100 tonnes.

It has now been found, surprisingly, that the catalyst according to the invention, consisting of catalytically active metal foam bodies, achieves the object of the invention.

“Metal foams” is to be understood as meaning rigid metallic foams having a high porosity and numerous interconnections between regions of solid material having an irregular structure. Such metal foams are also known as “cellular metals”, the term “metal foams” being more widespread. The term “metal foams” is well established in the technical literature and is more particularly elucidated for example in Ullmann's Encyclopedia of Industrial Chemistry in the chapter entitled “Metallic Foams”, published online on 15 Jul. 2012, DOI: 10.1002/14356007.c16_c01.pub2.

“Catalytically active metal foam bodies” in the context of this invention is to be understood as meaning metal foam bodies comprising no additional coating that could potentially bring about a narrowing of the pores in the metal foam bodies. Catalytically active metal foam bodies in the context of this invention are catalytically active in the target reaction—here: in the conversion of IPN to IPDA—without additional coatings, catalytically active or otherwise.

The invention provides a one-stage process for preparing 3-aminomethyl-3,5,5-trimethylcyclohexanol by hydrogenation of isophoronenitrile (IPN) with hydrogen over a catalyst, in the presence or absence of solvents,

wherein the catalyst has the following properties:

### I.

the catalyst is present in the form of catalytically active metal foam bodies and has a bulk density of not more than 0.8 kg/1, and

### II.

the catalyst in the activated state in its entirety has the following composition in weight percent (wt %), wherein the proportions add up to 100 wt %, based on the metals present:
cobalt: 70 to 99.8 wt %
aluminium: 0.02 to 15 wt %
and also one or more elements selected from the group consisting of chromium, nickel and iron:
0 to 15 wt %,
   and
   III. the catalytically active metal foam bodies have an average particle size of 1 up to a maximum of 70 mm,
   and
   IV. the catalytically active metal foam bodies exhibit a porous foam structure, wherein the macroscopic pores have sizes in the range of 100 to 5000 μm, preferably 200 to 2500 μm, particularly preferably 400 to 1200 μm.

### Catalyst:

### Constituents

The catalyst according to the invention includes activated metal catalysts. Activated metal catalysts are also fundamentally known to a person skilled in the art as catalysts of the “Raney type” or simply “Raney catalysts”. They are generally produced by forming an at least binary metal alloy of at least one optionally catalytically active metal component and at least one leachable metal component and subsequently leaching out the leachable metal component.

The catalyst in the activated state in its entirety has the following composition in weight percent (wt %), wherein the proportions add up to 100 wt %, based on the metals present:

cobalt: 70 to 99.8 wt %
aluminium: 0.02 to 15 wt %
and also one or more elements selected from the group consisting of chromium, nickel and iron:
0 to 15 wt %.

### PREFERRED EMBODIMENT

The catalyst according to the invention comprises 70 to 99.8 wt %, preferably 70 to 95 wt %, especially preferably 80 to 90 wt % cobalt and 0.02 to 15 wt %, preferably 4 to 13 wt %, especially preferably 6 to 12 wt % aluminium and preferred embodiments of the catalyst according to the invention also comprise 0 to 15 wt %, preferably 0.05 to 8 wt %, particularly preferably 0.1 to 3.5 wt % and especially preferably 0.2 to 2.5 wt % of one or more elements selected from the group consisting of chromium, nickel and iron. Particular preference is given to chromium and iron. The ranges specified may be combined with one another as desired.

Preferred embodiments of the catalyst according to the invention further comprise 0 to 10 wt % doping metals and/or promoters for increasing activity, selectivity and/or service life. Preferably present are one or more doping metals, selected from the group consisting of platinum (Pt), palladium (Pd), rhodium (Rh), ruthenium (Ru), osmium (Os), iridium (Ir), molybdenum (Mo), tungsten (Wo), manganese (Mn), rhenium (Re), copper (Cu), silver (Ag) and gold (Au), in a concentration range of 0.05 to 10 wt %, particularly preferably 0.2 to 3.5 wt %.

The catalyst in the activated state in its entirety particularly preferably has the following composition in weight percent (wt %), wherein the proportions add up to 100 wt %, based on the metals present:

cobalt: 80 to 90 wt %
aluminium: 6 to 12 wt %
and also one or more elements selected from the group consisting of chromium, nickel and iron:
0.05 to 8 wt %.

The catalyst in the activated state in its entirety especially preferably has the following composition in weight percent (wt %), wherein the proportions add up to 100 wt %, based on the metals present:

cobalt: 80 to 90 wt %
aluminium: 6 to 12 wt %
and also one or more elements selected from the group consisting of chromium, nickel and iron:
0.1 to 3.5 wt %.

The catalyst according to the invention is obtained by activating the cobalt metal foam modified with aluminium and optionally with further metals. For this purpose, at least a portion of the aluminium is chemically leached out of the alloy material. For this purpose, aqueous basic solutions are used, preferably alkali metal hydroxide solutions.

The catalyst according to the invention is produced by leaching aluminium from an at least binary metal alloy comprising aluminium (Al) and cobalt (Co) and optionally the further constituents.

### Properties

### Bulk Density

The catalyst according to the invention has a bulk density of not more than 0.8 kg/l, preferably from 0.1 to 0.7 kg/l, particularly preferably from 0.2 to 0.6 kg/l.

The bulk density of the catalyst according to the invention is therefore distinctly lower than the “Raney catalysts” known from the prior art which are used to prepare IPDA from IPN.

Bulk density (d_{Sch}), sometimes also referred to as poured density of a solid, is the ratio of mass to volume of a mixture of a granular solid and air which fills the cavities between the particles. This parameter which is commonly used by those skilled in the art is determined by means of a measuring cylinder by determining the mass (M_{F}) of a defined bed volume of solid (V_{F}):

d_{Sch}=M_{F}/V_{F}

### Particle Sizes

The catalyst according to the invention consists of catalytically active metal foam bodies. These catalytically active metal foam bodies preferably have an average particle size of 1 up to a maximum of 70 mm. In principle, the optimal particle size is controllable and is matched to the conditions prevailing in the reactor used. The catalyst according to the invention is preferably used as a bulk material composed of catalytically active metal foam bodies, wherein the catalytically active metal foam bodies have a particle size of 1 to 50 mm, particularly preferably 1 to 30 mm and especially preferably 1 to 10 mm. The particles of a bulk material may therefore have identical or different sizes.

The average particle size for the particles having a size in the range of 5 μm to 125 mm can be determined by sieve analysis according to DIN 66165. Suitable methods and descriptions of sieve analysis are given in:

DIN 66165-1:1987-04 Particle size analysis; sieve analysis; general principles, and in DIN 66165-2:1987-04 Particle size analysis; sieve analysis; procedure.
Paul Schmidt, Rolf Körber, Matthias Coppers: Sieben and Siebmaschinen (Sieving and sieving machines): Grundlagen and Anwendung (Fundamentals and Applications). Wiley-VCH Verlag, 2003, ISBN 9783527302079, chapter 4.4: Analysesiebung (sieve analysis).
Jörg Hoffmann: Handbuch der Messtechnik (Handbook of measurement techniques). Hanser Verlag, 2007, ISBN 978-3-446-40750-3, chapter 3.12.16.2.1.

Alternatively, average particle size can be determined optically, by means of a microscope, in which a number average of at least 100 individual values should be determined.

### BET Surface Area

The catalyst according to the invention has a BET surface area of 1 to 200 m²/g, preferably 10 to 120 m²/g, particularly preferably 70 to 100 m²/g. The specific surface area, also referred to as BET surface area for simplicity, is determined in accordance with DIN 9277 by nitrogen adsorption according to the Brunauer-Emmett-Teller process as described in J. Am. Chem. Soc. 1938, Vol. 60, pp. 309-319.

### Pore Sizes

The catalyst according to the invention consists of catalytically active metal foam bodies. Metal foam structures comprising many cavities may for example be formed by action of gases on a liquefied metal and subsequent cooling. A further option for achieving such structures is using organic foam structures as a template (basis) for the application of a metal and subsequently removing the organic template by incineration.

The catalytically active metal foam bodies, from which the catalyst according to the invention is composed, preferably exhibit a porous foam structure, wherein the macroscopic pores have sizes in the range of 100 to 5000 μm, preferably 200 to 2500 μm, particularly preferably 400 to 1200 μm. The size of the macroscopic pores may be determined using for example a method described in “The Guide 2000 of Technical Foams”, book 4, part 4, pages 33-41. The size of the macroscopic pores may be determined by optical measurement of the pore diameter of a selected pore. This measurement is repeated for at least 100 different pores, and an average value of the pore diameter is calculated therefrom as analysis result.

### General Method for Preparation of the Catalyst

To prepare a catalyst according to the invention, a commercially available cobalt metal foam is sprayed with an adhesion promoter, coated with aluminium powder and optionally further metal powders such as iron and/or nickel and/or chromium for example, and the material thus obtained is subjected to heat treatment. Then a comminution, separation and/or shaping of the alloy material obtained after the heat treatment is carried out. The catalytically active metal foam bodies, of which the catalyst according to the invention consists, are produced from the alloy foam bodies thus obtained by leaching at least a portion of the aluminium contained therein.

The cobalt metal foam to be used to prepare the catalyst according to the invention is preferably used in sheet form with edge lengths up to 500 mm and a thickness of not more than 5 mm. In order to improve the adhesion of aluminium powder and optionally further metal powders to the cobalt metal foam, this is initially treated with an adhesion promoter. Any adhesion promoter which improves adhesion between metals and organic materials may be employed. Polyethyleneimine solution for example is suitable.

After application of the aluminium powder and optionally further metal powder to the cobalt metal foam, the material is subjected to a heat treatment in the temperature range of 500 to 1000° C., preferably 600 to 800° C., wherein firstly moisture and organic residues from the aforementioned coating process are removed and subsequently aluminium is at least partially liquefied and is alloyed into the cobalt foam structure. The heat treatment is effected in an atmosphere of oxygen-free inert gas to prevent formation of disruptive oxidic layers.

The material thus obtained, a cobalt metal foam modified with aluminium and optionally further metals, is then optionally comminuted, separated and/or subjected to an appropriate shaping. The comminution and separation of the modified cobalt metal foam may be effected, for example, by laser cutting or laser beam cutting. The resulting material pieces (particles) preferably have a cuboid or parallelepipedal shape having a maximum edge length of not more than 50 mm and form a bulk material in their totality.

In the next step, the catalyst according to the invention is obtained by activating the cobalt metal foam modified with aluminium and optionally with further metals. For this purpose, at least a portion of the aluminium is chemically leached out of the alloy material. For this purpose, aqueous basic solutions are used, preferably alkali metal hydroxide solutions selected from the group consisting of solutions of sodium hydroxide, potassium hydroxide or lithium hydroxide, particular preference being given to aqueous sodium hydroxide solution. The concentration of the aqueous alkali metal hydroxide solution employed in the catalyst preparation can generally be between 0.1 and 60 wt % alkali metal hydroxide. The leaching out of the aluminium is preferably effected with a 5 to 50 wt %, particularly preferably 5 to 35 wt %, aqueous sodium hydroxide solution at a temperature of 20° C. to 100° C., preferably at 40° C. to 85° C., particularly preferably at 50° C. to 80° C. The leaching times to be used here, i.e. the reaction times of the sodium hydroxide solution with the cobalt metal foam modified with aluminium, depend on the aluminium content to be set in the end product, as well as the other reaction conditions mentioned above, and may be between 2 and 240 minutes.

The catalyst according to the invention generated by modification and activation retains the macroscopic foam structure of the originally used cobalt metal foam. The at least partial leaching out of the aluminium is effected in near-surface regions where a highly porous, catalytically active cobalt structure is generated. The BET surface area of the catalyst according to the invention is preferably greater than that of the cobalt metal foam used.

To improve the activity, selectivity and/or service life of the catalyst according to the invention, various dopants and/or promoters may be added. This may be effected by alloying a dopant metal into the cobalt metal foam used in the catalyst preparation and/or by wet-chemical post-treatment of the catalyst according to the invention. For example, the dopant metal can be applied by precipitation or reductive decomposition from preferably aqueous solution of a suitable precursor. One or more dopant elements are preferably applied, selected from the group consisting of platinum (Pt), palladium (Pd), rhodium (Rh), ruthenium (Ru), osmium (Os), iridium (Ir), molybdenum (Mo), tungsten (Wo), manganese (Mn), rhenium (Re), copper (Cu), silver (Ag) and gold (Au).

The catalyst according to the invention is used for the preparation of isophorone amino alcohol by hydrogenation.

The hydrogenation is effected in one stage in batch autoclaves or fixed bed reactors. Suitable reactor types are, for example, stirred tanks, shaft furnaces, tray reactors or shell and tube reactors.

The process can be conducted in batchwise or continuous mode.

The hydrogenation is effected typically at temperatures between 20 and 200° C., preferably 20 and 150° C., more preferably 40 and 130° C., and pressures of 0.3 to 50 MPa, preferably 0.3 to 30 MPa, more preferably 5 to 15 MPa. The ranges for pressure and temperature can be combined as desired. The hydrogenation can be conducted in the presence or absence of solvent. Suitable solvents include ethers, cyclic and linear hydrocarbons and alcohols, preference being given to using methanol, ethanol or THF.

The hydrogen required for the hydrogenation may be supplied to the reactor either in excess, for example at up to 10,000 molar equivalents, or merely in an amount such that the hydrogen consumed by reaction, and the portion of the hydrogen that leaves the reactor dissolved in the product stream, is replenished. In the case of a continuous mode of operation, the hydrogen may be supplied in cocurrent or countercurrent.

The required volume of the hydrogenation catalysts to be used is guided by the LHSV-value (liquid hourly space velocity), which is dependent on the operating pressure, the temperature, the concentration and the catalyst activity and has to be maintained in order to ensure maximum completeness of hydrogenation of the IPN used. Typically, the LHSV-value in the case of use of the mixture of IPN and hydrogen, the use of which is preferred, is between 0.1 and 8 liters of solution per liter of catalyst and hour, preferably between 1 and 4 Lₛₒₗ L_{cat}⁻¹ h⁻¹.

The hydrogenation is preferably performed continuously in fixed bed reactors, particularly preferably continuously in fixed bed reactors which are operated in trickle mode or liquid phase mode.

The reaction mixture leaving the hydrogenation is purified further by the customary methods, in order to obtain an IPAA with the desired quality. Any standard separation methods, for example distillation, flash evaporation, crystallization, extraction, sorption, permeation, phase separation or combinations of the above, may be employed here. The purification may be performed continuously, batchwise, as a single- or multi-stage procedure, under vacuum or under pressure.

Purification is preferably achieved by distillation under pressure and/or under vacuum in a plurality of steps. Any desired distillation columns with or without internals may be used to this end, for example dephlegmators, dividing walls, unordered internals or random packings, ordered internals or structured packings, or trays with or without forced flow.

### EXAMPLES

### Preparation of the Catalyst

A cobalt metal foam commercially available in rolls and having a thickness of 1.9 mm, a width of 300 mm and an average pore size of 580 μm was sprayed with a commercially available adhesion promoter solution, coated with aluminium powder and subjected to heat treatment at 700° C. After cooling, the material thus obtained was cut with a laser into square pieces having an edge length of 4 mm×4 mm and a thickness of 1.9 mm.

To achieve catalytic activation the resulting bulk material was arranged in a fixed bed and subjected to wet-chemical aftertreatment by pumping a 10 wt % aqueous sodium hydroxide solution therethrough at 60° C. for a period of 60 minutes. The material was subsequently washed with water until a pH of the wash solution after pumping through the fixed bed of less than 10 had been reached.

The composition of the catalytically active metal foam bodies thus obtained was analysed by ICP-OES. The results are compiled in Table 1 below:

TABLE 1

| Cobalt content | Aluminium content | Bulk density |
|---|---|---|
| 97.4 wt % | 2.09 wt % | 0.5 kg/L |

Preparation of IPAA with the Inventive Catalyst

The inventive catalyst was tested for its catalytic efficacy in the preparation of 3-aminomethyl-3,5,5-trimethylcyclohexanol (isophorone amino alcohol, IPAA) from 3-cyano-3,5,5-trimethylcyclohexanone (isophoronenitrile, IPN) in a batchwise process in a laboratory autoclave.

For the performance of the individual experiments, the reaction solution composed of 10% by weight of IPN and 90% by weight of THF was initially charged in the reactor. 150 ml of the catalyst were in a special catalyst basket. After the heating to the desired reaction temperature, the desired reaction pressure was established by adding hydrogen. The hydrogenation of IPN with the catalysts according to the invention was studied at a temperature of 100° C. at a pressure of 50 bar.

The product solution was analysed by gas chromatography for IPN, IPAA and corresponding secondary components. The results are listed in Table 2.

TABLE 2

| Temperature | Pressure | Reaction time | IPAA yield/GC % | Conversion |
|---|---|---|---|---|
| 100° C. | 50 bar | 5 h | 81.4% | 100% |

1. A one-stage process for preparing 3-aminomethyl-3,5,5-trimethylcyclohexanol by hydrogenation of isophoronenitrile (IPN) with hydrogen over a catalyst, in the presence or absence of solvents,
wherein the catalyst has the following properties:
I. the catalyst is present in the form of catalytically active metal foam bodies and has a bulk density of not more than 0.8 kg/1, and
II. the catalyst in the activated state in its entirety has the following composition in weight percent (wt %), wherein the proportions add up to 100 wt %, based on the metals present:
cobalt: from 70 to 99.8 wt %
aluminium: from 0.02 to 15 wt %
and also one or more elements selected from the group consisting of chromium, nickel and iron: 0 to 15 wt %, and
III. the catalytically active metal foam bodies have an average particle size of from 1 up to a maximum of 70 mm, and
IV. the catalytically active metal foam bodies exhibit a porous foam structure, wherein the macroscopic pores have sizes in the range of from 100 to 5000 μm.

2. The process according to claim 1, wherein the catalyst has a bulk density of from 0.1 to 0.7 kg/l.

3. The process according to claim 1, wherein the catalyst comprises from 70 to 95 wt % cobalt and from 0.02 to 15 wt % aluminium and from 0 to 15 wt % of one or more elements selected from the group consisting of chromium, nickel and iron.

4. The process according to claim 1, wherein the catalyst in the activated state in its entirety has the following composition in weight percent (wt %), wherein the proportions add up to 100 wt %, based on the metals present:
cobalt: from 80 to 90 wt %
aluminium: from 6 to 12 wt %
and also one or more elements selected from the group consisting of chromium, nickel and iron: from 0.05 to 8 wt %.

5. The process according to claim 1, wherein the catalyst in the activated state in its entirety has the following composition in weight percent (wt %), wherein the proportions add up to 100 wt %, based on the metals present:
cobalt: from 80 to 90 wt %
aluminium: from 6 to 12 wt %
and also one or more elements selected from the group consisting of chromium, nickel and iron: from 0.1 to 3.5 wt %.

6. The process according to claim 1, wherein the catalyst comprises 0 to 10 wt % doping metals and/or promoters for increasing activity, selectivity and/or service life.

7. The process according to claim 1, wherein the catalytically active metal foam bodies have a particle size of from 1 up to 50 mm, particularly preferably 1 up to 30 mm and especially preferably 1 to not more than 10 mm.

8. The process according to claim 1, wherein the catalytically active metal foam bodies of the catalyst have macroscopic pores of sizes in the range of from 100 to 5000 μm.

9. The process according to claim 1, wherein the catalyst has a BET surface area of from 10 to 120 m²/g.

10. The process according to claim 1, wherein the hydrogenation is performed at a temperature of from 20 to 200° C. and at a pressure of from 0.3 to 50 MPa.

11. The process according to claim 1, wherein the hydrogenation is effected in one stage in batch autoclaves or fixed bed reactors.

12. The process according to claim 1, wherein the hydrogen required for the hydrogenation is supplied to the reactor either in excess, preferably at up to 10,000 molar equivalents, or in such an amount that the hydrogen consumed by reaction and the portion of the hydrogen which leaves the reactor dissolved in the product stream is replenished.

13. The process according to claim 1, wherein the hydrogenation is effected continuously in fixed bed reactors.

14. The process according to claim 1, wherein the hydrogenation is conducted continuously in fixed bed reactors which are operated in trickle mode or liquid phase mode.

15. The process according to claim 1, wherein the reaction mixture leaving the hydrogenation is purified in one or more stages, and the 3-(aminomethyl)-3,5,5-trimethylcyclohexanol is obtained.

16. The process according to claim 1, wherein the catalyst has a bulk density of from 0.2 to 0.6 kg/l.

17. The process according to claim 1, wherein the catalyst comprises from 80 to 90 wt % cobalt and from 6 to 12 wt % aluminium and from 0.2 to 2.5 wt % of one or more elements selected from the group consisting of chromium, nickel and iron.

18. The process according to claim 1, wherein the catalytically active metal foam bodies have a particle size of from 1 up to 10 mm.

19. The process according to claim 1, wherein the catalytically active metal foam bodies of the catalyst have macroscopic pores of sizes in the range of from 400 to 1200 μm.

20. The process according to claim 1, wherein the catalyst has a BET surface area of 70 to 100 m²/g.
